(19) United States
(12) Patent Application Publication
(10) Pub. No.: US 2020/0397577 A1
(43) Pub. Date: Dec. 24, 2020

(54) TAVR Guidewire

(71) Applicant: St. Jude Medical, Cardiology Division, Inc., St. Paul, MN (US)

(72) Inventors: Yousef F. Alkhatib, Edina, MN (US); Michael Shane Morrissey, St. Paul, MN (US); Jay Reimer, Saint Paul, MN (US); Kristopher Henry Vietmeier, Monticello, MN (US); Peter J. Ness, Minneapolis, MN (US)

(73) Assignee: St. Jude Medical, Cardiology Division, Inc., St. Paul, MN (US)

(21) Appl. No.: 16/904,661

(22) Filed: Jun. 18, 2020

Related U.S. Application Data
(60) Provisional application No. 62/864,732, filed on Jun. 21, 2019.

(51) Int. Cl. A61F 2/24; A61F 2/95

(52) U.S. Cl. CPC A61F 2/2436; A61F 2/9517

(57) ABSTRACT

A guidewire may be configured for insertion into a heart of a patient during a procedure such as a transcatheter aortic valve replacement procedure. The guidewire may include a proximal end and a distal end portion. The distal end portion may include (i) a leading section, (ii) a loop structure at a terminal distal end of the guidewire, and (iii) a transition section extending between the leading section and the loop structure. In the absence of applied forces, the leading section is not tangential to the loop structure. With such a configuration, the guidewire may avoid contact with the ventricular septum of the heart when the loop structure is seated within the left ventricle, which may mitigate potential interference with conduction pathways in the ventricular septum, which may in turn mitigate the need for a pacemaker.

### CROSS-REFERENCE TO RELATED APPLICATIONS

This application claims the benefit of the filing date of U.S. Provisional Patent Application No. 62/864,732, filed Jun. 21, 2019, the disclosure of which is hereby incorporated by reference herein.

### BACKGROUND OF THE DISCLOSURE

The present disclosure relates to transcatheter aortic valve replacement (“TAVR”) devices and methods and, in particular, guidewires for use in such procedures. However, it should be understood that the guidewires described herein may be useful in other procedures in which transcatheter entry into the heart is desired.

Prosthetic heart valves that are collapsible to a relatively small circumferential size can be delivered into a patient less invasively than valves that are not collapsible. For example, a collapsible valve may be delivered into a patient via a tube-like delivery apparatus such as a catheter, a trocar, a laparoscopic instrument, or the like. This collapsibility can avoid the need for a more invasive procedure such as full open-chest, open-heart surgery.

Collapsible prosthetic heart valves typically take the form of a valve structure mounted on a stent. There are two types of stents on which the valve structures are ordinarily mounted: a self-expanding stent and a balloon-expandable stent. To place such valves into a delivery apparatus and ultimately into a patient, the valve must first be collapsed or crimped to reduce its circumferential size.

Generally, when implanting a collapsible prosthetic aortic valve into a patient, one of the first steps is to advance a guidewire into the left ventricle. Once the guidewire is in the desired position, other devices, such a delivery device that houses the prosthetic heart valve in a collapsed condition, may be advanced over the guidewire, with the guidewire helping to guide the device to the desired site of implantation.

However, when conventional guidewires are passed through the vasculature and into the left ventricle, for example during a TAVR procedure, certain problems may arise. During the process of advancing the guidewire into the left ventricle, the guidewire may contact the ventricular septum, which may result in interference with the conduction system of the heart. Similarly, while manipulating a guidewire positioned within the left ventricle, or while advancing or retracting other components of a delivery system over the guidewire positioned within the left ventricle, undesirable contact between the guidewire and the ventricular septum may occur. Interfering with the conduction system of the heart, for example via contact between the guidewire and the ventricular septum, may require a pacemaker to be implanted during or following the TAVR procedure in order to compensate for the conduction interference caused during the procedure. Thus, it would be desirable to have guidewires, and methods of using guidewires, that reduce the likelihood of causing conduction interference via contact with the ventricular septum.

### BRIEF SUMMARY

According to a first aspect of the disclosure, a guidewire for insertion into a heart includes a proximal end and a distal end portion. The distal end portion may include (i) a leading section, (ii) a loop structure at a terminal distal end of the guidewire, and (iii) a transition section extending between the leading section and the loop structure. In the absence of applied forces, the leading section is not tangential to the loop structure.

According to another aspect of the disclosure, a method of positioning a guidewire within a heart includes advancing a distal end portion of the guidewire into a left or right ventricle of the heart until a loop structure at a terminal distal end of the guidewire is seated within the left or right ventricle. The distal end portion of the guidewire may include a leading section and a transition section extending between the leading section and the loop structure. When the loop structure is seated within the left or right ventricle, an entire length of the leading section positioned between a native valve annulus of the heart and the transition section of the guidewire may be out of contact with a ventricular septum separating the left ventricle from the right ventricle of the heart.

### BRIEF DESCRIPTION OF THE DRAWINGS

FIG. 1 is a side view of a delivery device for a prosthetic heart valve assembled to an introducer.

FIG. 2A is a top plan view of a portion of an operating handle for the delivery device of FIG. 1, shown with a partial longitudinal cross-section of the distal portion of a transfemoral catheter assembly.

FIG. 2B is a side view of the handle of FIG. 2A.

FIG. 3 is a schematic representation of a human heart and associated blood vessels.

FIG. 4 is a schematic view of the distal end of a guidewire according to the prior art.

FIG. 5 is a schematic view of the distal end of the guidewire of FIG. 4 positioned within the left ventricle.

FIG. 6 is a schematic view of the distal end of a guidewire according to an embodiment of the disclosure.

FIG. 7 is a schematic view of the distal end of the guidewire of FIG. 5 positioned within the left ventricle.

FIGS. 8-14 are front views of further embodiments of a distal end of a guidewire.

FIGS. 15-17 are side views of further embodiments of a distal end of a guidewire.

FIGS. 18A-B are side views of an embodiment of a distal end of a guidewire having a generally spherical tip.

FIG. 19 is a side view of an embodiment of a distal end of a guidewire having a tip with a directional bulge.

FIG. 20 is a highly schematic cross-section of a distal end of a guidewire positioned through the aortic arch and within the native aortic valve annulus.

FIG. 21 is a highly schematic cross-section of a distal end of another embodiment of a guidewire positioned through the aortic arch and through the native aortic valve annulus.

FIG. 22 is a highly schematic cross-section of a distal end of another embodiment of a guidewire positioned through the aortic arch, the left ventricle the left atrium, and into the left atrial appendage.

FIG. 23 is a highly schematic cross-section of a distal end of another embodiment a guidewire positioned in the left ventricle.

FIG. 24 is a transverse cross section of a composite guidewire according to another embodiment of the disclosure.

FIG. 25A is a highly schematic cross-section of a guidewire and delivery device positioned within a heart.

FIG. 25B is a view of an alternate embodiment of the guidewire of FIG. 25A.

FIG. 26A is a highly schematic cross-section of a guidewire and delivery device positioned within a heart.

FIG. 26B is a top-down view of an alternate embodiment of a distal loop of the guidewire of FIG. 26A.

FIG. 26C is a side view of an alternate version of the guidewire of FIG. 26A with an overlying sheath positioned thereon.

FIG. 27A is a highly schematic cross-section of a guidewire and delivery device positioned within a heart, with a tip of the guidewire in an expanded condition.

FIG. 27B is a side view of the tip of the guidewire of FIG. 27A in a collapsed condition.

FIG. 27C is a side view of an alternate version of the tip of the guidewire of FIG. 27B in a collapsed condition.

FIGS. 27D-E are side views of a delivery device with a distal tip similar to that of the distal tip of the guidewire shown in FIGS. 27A-B, in collapsed and expanded conditions, respectively.

### DETAILED DESCRIPTION

As used herein, the term “proximal,” when used in connection with a guidewire and/or delivery device, refers to an end of the device closer to the user of the device when the device is being used as intended. On the other hand, the term “distal,” when used in connection with a guidewire and/or delivery device, refers to an end of the device farther away from the user. In the figures, like numbers refer to like or identical parts. As used herein, the terms “substantially,” “generally,” “approximately,” and “about” are intended to mean that slight deviations from absolute are included within the scope of the term so modified. When ranges of values are described herein, those ranges are intended to include sub-ranges. For example, a recited range of 1 to 10 includes 2, 5, 7, and other single values, as well as all sub ranges within the range, such as 2 to 6, 3 to 9, 4 to 5, and others.

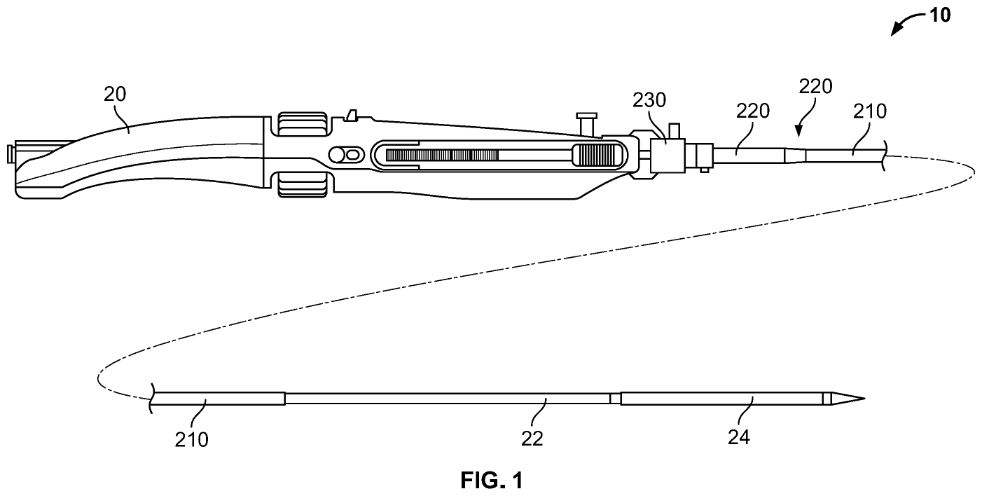

FIG. 1 shows a prosthetic heart valve delivery device 10 assembled to an introducer 200. Generally, delivery device 10 includes an operating handle 20 coupled to an outer catheter shaft 22 extending through introducer 200. The delivery device 10 may also include a distal sheath 24 for holding a prosthetic heart valve therein. Introducer 100 may generally include a hollow distal sheath 210 connected to a proximal sheath 220, which in turn is connected to a housing 230.

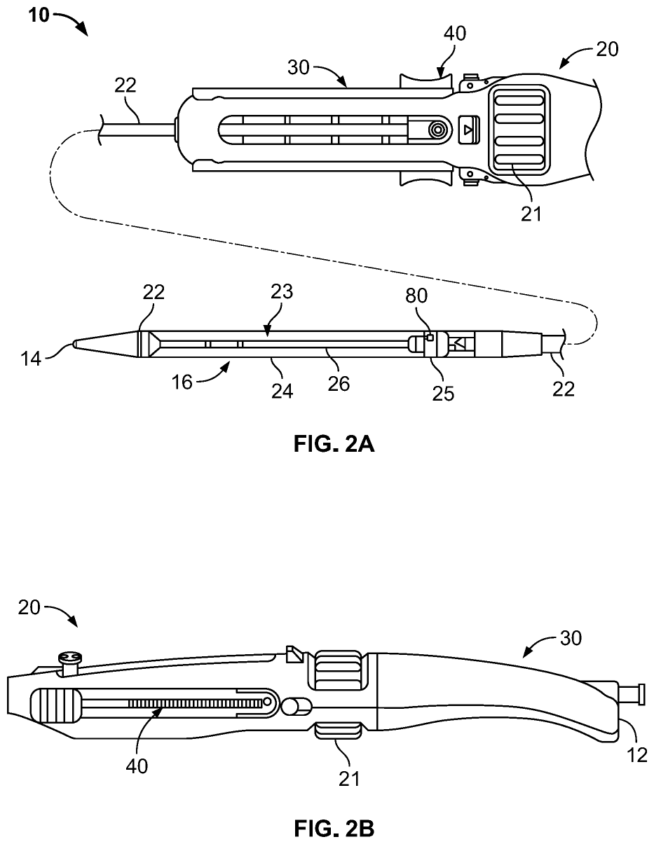

Referring now to FIGS. 2A-2B, delivery device 10 includes catheter assembly 16 for delivering the heart valve to, and deploying the heart valve at, a target location, and operating handle 20 for controlling deployment of the valve from the catheter assembly. Delivery device 10 extends from proximal end 12 (FIG. 2B) to atraumatic tip 14 at the distal end of catheter assembly 16. Catheter assembly 16 is adapted to receive a collapsible prosthetic heart valve (not shown) in compartment 23 defined around inner shaft 26 and covered by distal sheath 24.

Inner shaft 26 may extend through operating handle 20 to atraumatic tip 14 of delivery device 10, and may include retainer 25 affixed thereto at a spaced distance from tip 14 and adapted to hold a collapsible prosthetic valve in compartment 23. Retainer 25 may have recesses 80 therein that are adapted to hold corresponding retention members of the valve.

Distal sheath 24 surrounds inner shaft 26 and is slidable relative to inner shaft 26 such that it can selectively cover or uncover compartment 23. Distal sheath 24 is affixed at its proximal end to outer shaft 22, the proximal end of which is connected to operating handle 20. The distal end 27 of distal sheath 24 abuts atraumatic tip 14 when the distal sheath is fully covering the compartment 23, and is spaced apart from the atraumatic tip when compartment 23 is at least partially uncovered.

Operating handle 20 is adapted to control deployment of a prosthetic valve located in compartment 23 by permitting a user to selectively slide outer shaft 22 proximally or distally relative to inner shaft 26, thereby respectively uncovering or covering compartment 23 with distal sheath 24. The proximal end of inner shaft 26 may be connected in a substantially fixed relationship to outer housing 30 of operating handle 20, and the proximal end of outer shaft 22 is affixed to carriage assembly 40 that is slidable along a longitudinal axis of the handle housing, such that a user can selectively slide outer shaft 22 relative to inner shaft 26 by sliding carriage assembly 40 relative to the handle housing. For example, a user may rotate deployment actuator 21 to move carriage assembly 40 proximally, thus moving outer shaft 22 and distal sheath 24 proximally to uncover a prosthetic heart valve positioned within compartment 23 in the collapsed condition. As distal sheath 24 begins to clear the prosthetic heart valve, the prosthetic heart valve begins to expand to an expanded condition so that it may be fixed within the native heart valve annulus of interest. One example of a prosthetic heart valve that may be suitable for use with delivery system 10 is described in greater detail in U.S. Pat. No. 9,039,759, the disclosure of which is hereby incorporated by reference herein.

Delivery device 10 may include a guidewire lumen (not illustrated) passing partially or entirely therethrough. The guidewire lumen may extend to distal tip 14. As noted above, prior to advancing delivery device 10 into the patient, a guidewire may be advanced to the site of implantation to aid in guiding the delivery device 10 to the desired site of implantation. In a TAVR procedure, the guidewire may be advanced into the left ventricle. Once the guidewire is positioned in the left ventricle, the distal tip 14 of delivery system 10 may be threaded over a proximal end of the guidewire, with the guidewire guiding the distal end of the delivery device 10 toward the left ventricle during advancement of the delivery system 10. Prior to describing the guidewire in more detail, a brief description of a typical human heart is provided below.

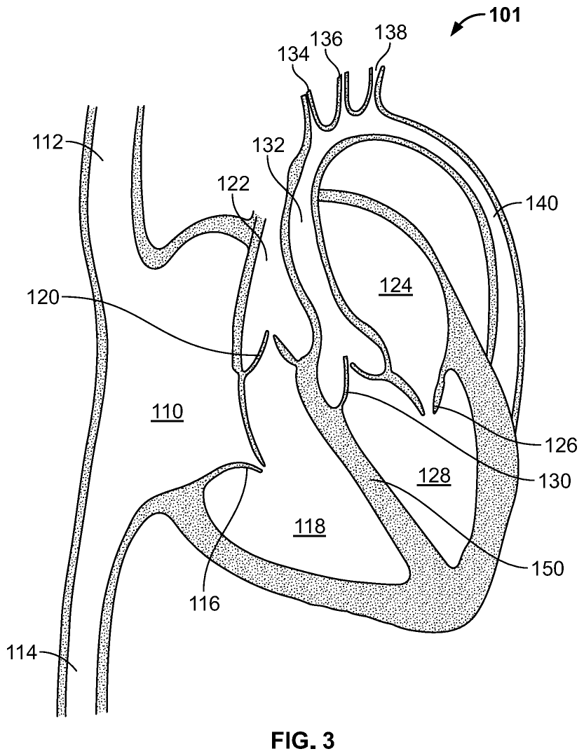

FIG. 3 is a schematic view of a typical human heart 101 and selected blood vessels leading to or from the heart. Briefly, deoxygenated blood enters the right atrium 110 from the superior vena cava 112 and the inferior vena cava 114. The right atrium 110 contracts to force blood through the tricuspid valve 116 and into the right ventricle 118. The right ventricle 118 then contracts to force blood through the pulmonary valve 120 into the pulmonary artery 122 which transports the blood to the lungs to become oxygenated. Oxygenated blood then returns from the pulmonary veins (not illustrated) and flows into the left atrium 124. The left atrium 124 contracts and forces blood through the mitral valve 126 and into the left ventricle 128. The left ventricle 128 contracts to force blood through the aortic valve 130 and into the ascending aorta 132. Blood is transported from the ascending aorta 132 to the rest of the body through a variety of other vessels such as the brachiocephalic artery 134, the left common carotid artery 136, the left subclavian artery 138, and the descending aorta 140. The left ventricle 128 and right ventricle 118 are separated by a ventricular septum 150. As noted above, if the ventricular septum 150 is contacted during a cardiac procedure, for example by a delivery device or related accessory, interference with the conduction of the heart 101 may occur, which may lead to ongoing conduction issues, including improper pacing of the heart 101, if the conduction issues are not rectified, for example via implantation of a pacemaker.

If the aortic valve 130 is not functioning properly, a prosthetic aortic valve may be implanted within the native aortic valve annulus, with the prosthetic heart valve taking over the function of the malfunctioning native aortic valve 130. Although various delivery routes are possible for a TAVR procedure, a common route is through the femoral artery. In such a transfemoral access route, the delivery device 10 may be passed into the femoral artery, and advanced through the descending aortic valve 140, around the aortic arch, down the ascending aorta 132, and into a desired position within or adjacent to the native aortic valve 130. With the delivery device 10 in the desired position, the delivery device 10 may be manipulated as described above to release a self-expanding prosthetic aortic valve from the delivery device, allowing the prosthetic aortic valve to expand within the native aortic valve 130 to take over for the malfunctioning native aortic valve 130. In other examples, the prosthetic aortic valve may be forced to expand, for example by inflating a balloon over which the prosthetic heart valve is positioned. In most or all cases, as noted above, a guidewire is initially passed through the pathway described above and into the left ventricle 128, with the guidewire serving as a rail for other devices, including delivery device 10, to be passed over in later steps after the guidewire has been inserted.

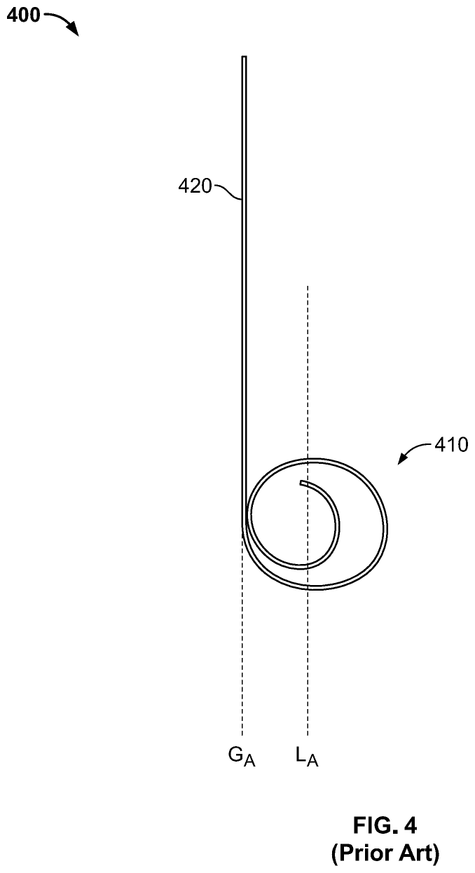

FIG. 4 is a schematic view of a guidewire 400 according to the prior art. FIG. 5 illustrates the distal end of guidewire 400 after it has been advanced through the vasculature and into the left ventricle 128. In particular, FIGS. 4-5 illustrate the distal end of guidewire 400, with the understanding that the proximal end and intermediate portions of guidewire 400 are not illustrated, but may be of any suitable length to reach from outside the patient, through the vasculature, into left ventricle 128. Guidewire 400 includes one or more loops, such as double loop 410, at the terminal distal end of the guidewire. Double loop 410 presents an atraumatic terminal end. In the prior art guidewire 400, the double loop 410 is formed generally in the same plane. The atraumatic terminal end of guidewire 400 reduces the likelihood of the guidewire 400 puncturing or perforating tissue, including the relatively fragile tissue of (and within) the left ventricle 128, as the guidewire 400 is being advanced through the vasculature and into the left ventricle 128. Typical guidewires such as guidewire 400 include a leading section 420 that is positioned just proximal to the double loop 410 and is designed to be substantially straight and substantially tangential of the beginning of the double loop 400. In other words, as shown in FIG. 4, the leading section 420 may extend along a guidewire axis G_{A} that is parallel to, but spaced apart from, a loop axis L_{A} that extends through a center portion of double loop 410. As shown in FIG. 5, when the double loop 410 of guidewire 400 is seated within the left ventricle 128, the above-described traditional design of guidewire 400 often results in the leading section 420 of the guidewire 400 being in contact with the ventricular septum 150. This contact, as described above, is undesirable, but may be difficult to avoid, especially with guidewire designs of the prior art. The contact between the leading section 420 of the guidewire 400 and the ventricular septum 150 may become even more pronounced as the guidewire 400 is manipulated, for example as delivery device 10 or another device is advanced over portions of the guidewire 400. Further, the beating of the heart 101 while the leading section 420 of the guidewire 400 is in contact with the ventricular septum 150 may tend to further exacerbate the contact, potentially causing further disruptions or interference with the natural conduction of the heart 101.

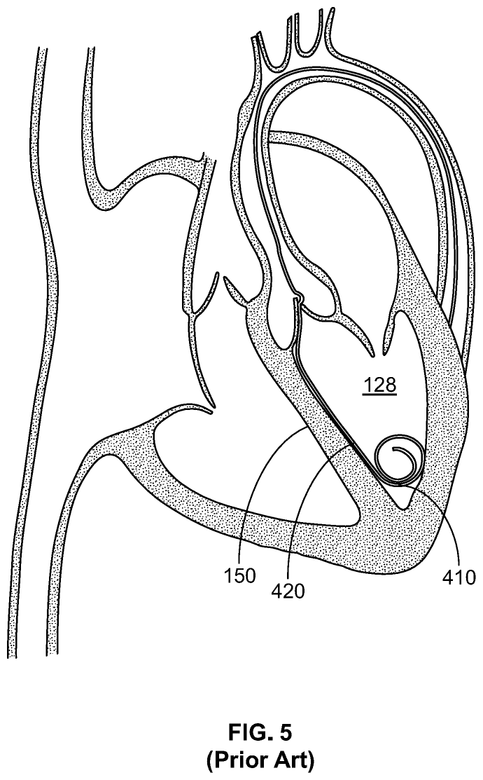

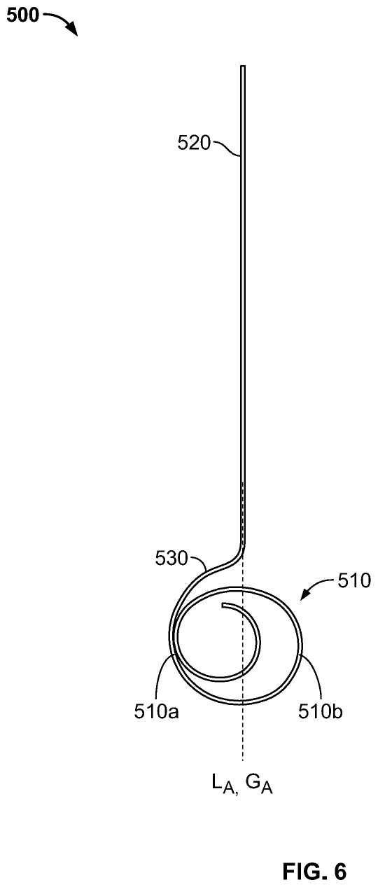

FIG. 6 is a schematic view of a guidewire 500 according to an embodiment of the disclosure. FIG. 7 illustrates the distal end of guidewire 500 after it has been advanced through the vasculature and into the left ventricle 128. In particular, FIGS. 6-7 illustrate the distal end of guidewire 500, with the understanding that the proximal end and intermediate portions of guidewire 500 are not illustrated, but may be of any suitable length to reach from outside the patient, through the vasculature, into left ventricle 128. Similar to guidewire 400, guidewire 500 may include one or more loops, such as double loop 510, at the terminal distal end of the guidewire. Double loop 510 may serve substantially the same purpose as double loop 410—presenting an atraumatic terminal end. However, whereas the leading section 420 of guidewire 400 is positioned substantially tangential to an outer loop portion of double loop 410, the leading section 520 of guidewire 500 is not. Rather, as shown in FIG. 6, leading section 520 extends in substantially a single, straight direction along a guidewire axis G_{A} that is substantially aligned with loop axis L_{A}. In other words, guidewire 500 includes a short transition section 530 extending between the distal end of leading section 520 and the beginning of double loop 510, so that the leading section 520 extends in a direction toward a center portion of double loop 510. As shown in FIG. 7, when the double loop 510 of guidewire 500 is seated within the left ventricle 128, the design of guidewire 500 allows for the leading section 520 of the guidewire 500 to be spaced apart from the ventricular septum 150. By spacing the leading section 520 of the guidewire 500 away from the ventricular septum 150, the likelihood of interfering with the conduction pathways of the ventricular septum 150 via contact with guidewire 500 are reduced or altogether eliminated.

Referring back to FIG. 6, although the guidewire axis G_{A} is illustrated as being co-extensive with loop axis L_{A}, the axes need not be perfectly aligned. In other words, the double loop 510 may include a first point 510a where transition section 530 begins to transition into double loop 510, and a second point 510b may be positioned diametrically opposed to the first point 510a. Stated otherwise, points 510a and 510b may be diametrically opposed portions of double loop 510 where lines that run tangent to points 510a and 510b are substantially parallel to guidewire axis G_{A}. The leading section 520 may extend along a guidewire axis G_{A} that extends anywhere between a line tangent to first point 510a and a line tangent to second point 510b. Although having guidewire axis G_{A} substantially centered between first point 510a and second point 510b may provide the greatest likelihood that the leading section will avoid contact with the ventricular septum 150, positioning the guidewire axis G_{A} anywhere between first point 510a and second point 510b may help mitigate undesirable contact between the leading section 520 and the ventricular septum 150.

Although guidewire 500 is illustrated with a double loop 510, it should be understood that the concepts described herein (including additional guidewire embodiments described below) may apply with substantially equal force to other embodiments, such as a guidewire with a single loop, or with three or more loops. Further, in some embodiments, guidewire 500 may terminate in less than a complete loop (i.e. a partial loop). In any of the above cases, it would be desirable that the guidewire axis G_{A} extend between opposing lateral sides of the outermost loop structure included at the distal terminal end of the guidewire. Guidewire 500 is described above as having a particular shape or shapes, including in the absence of applied forces. It should be understood that the description of guidewire 500 above may further be constrained by an omission or lack of any artificial means of maintaining the guidewire 500 in the described shape or configuration. For example, the shape(s) and/or configuration(s) of guidewire 500 are preferably capable of being maintained by only the structure of the guidewire 500 itself, without, for example, one or more additional overlying sheaths assisting in maintain the shape, or without other portions of the guidewire 500 forcing the particular shape, for example via cross-over points, knots, or the like.

Although guidewire 500 is described as being useful for a TAVR procedure using a transfemoral route, it should be understood that guidewire 500 may be useful for any procedure that requires a guidewire to be positioned within the left ventricle 128, particularly if access to the left ventricle 128 is obtained by advancing the guidewire through the aortic arch and through the aortic valve 130. It should further be understood that, although the inventive guidewires described herein are described as for use in the left ventricle, the guidewires may also be used in the right ventricle (e.g. after passing through the pulmonary valve) for similar purposes, including to help avoid contacting the ventricular septum. Further, the guidewires described herein may also be suitable for passing into the left ventricle via the mitral valve annulus, or into the right ventricle via the tricuspid valve annulus.

Further, it should be understood that the shape of the guidewires 400 and 500 are shown and described in connection with FIGS. 4 and 6 in an unbiased condition of the guidewire. In other words, the guidewires 400, 500 may have a degree of flexibility so that the shapes can be altered upon application of force. The shape of guidewire 500 described above is preferably the shape that the guidewire has in the absence of applied force. However, it should be understood that guidewire 500 is preferably stiff enough so that the generally described shape is maintained while the guidewire 500 is in the position shown in FIG. 7, whereby the leading section 520 of guidewire 500 maintains a distance from the ventricular septum 150 despite the normal forces experienced by the guidewire 500 during typical use.

Although guidewire 500 is illustrated as having one particular shape, it should be understood that guidewires according to the inventor may have various shapes, particularly at or near the looped end, and still provide benefits similar or identical to those described above in relation to guidewire 500.

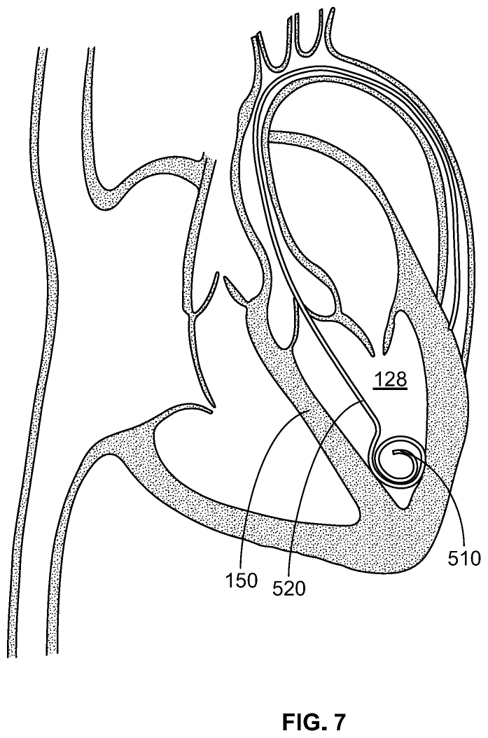

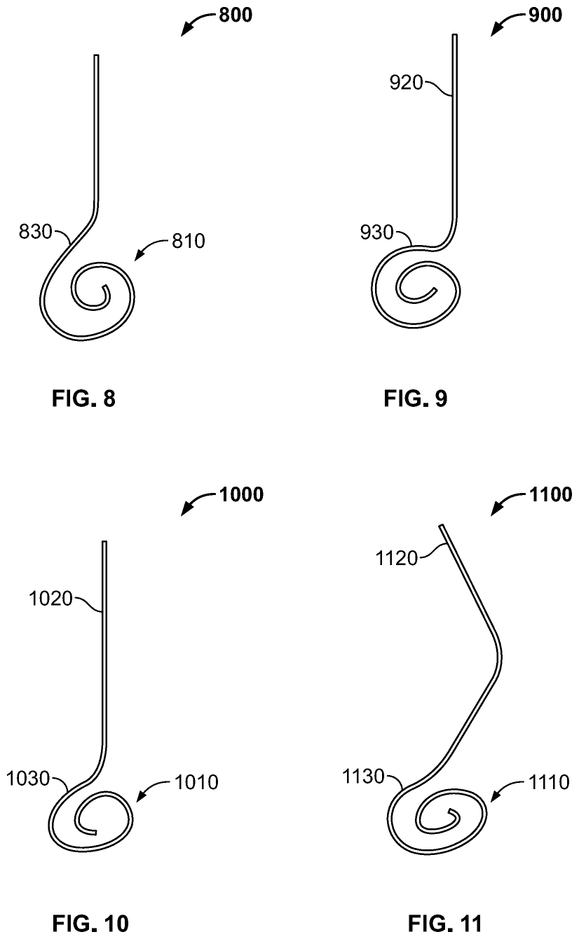

FIGS. 8-15 illustrate guidewires according to the present disclosure that have slight modifications, but maintain the overall purpose and general form, of guidewire 500. Each guidewire illustrated in FIGS. 8-15 may be similar or identical to guidewire 500, with certain exceptions described below. In other words, but for the differences described below, the description of guidewire 500 applies with equal force to the guidewires of FIGS. 8-15.

Guidewire 800 of FIG. 8 is substantially identical to guidewire 500, with the exception that the interior loop of the double loop 810 is not in contact with the exterior loop as illustrated in FIG. 6. Guidewire 900 of FIG. 9 is substantially identical to guidewire 800, with the exception that the transition section 930 is more pronounced. In other words, there is a more sudden or acute transition between leading section 920 and transition section 930, such that the leading section forms about a 90 degree angle where it transitions to the transition section 930, although it should be understood that the angle need not be sharp and may be rounded. This configuration of transition section 930 may provide additional assistance in avoiding contact with the ventricular septum. Guidewire 1000 is substantially identical to guidewire 800, with the main difference being that transition section 1030 is less rounded than the transition section 830 of guidewire 800. In other words, the transition section 1030 between double loop 1010 and leading end 1020 is mostly straight with relatively less curvature than the transition section 830 of guidewire 800. This configuration of transition section 1030 may provide additional assistance in avoiding contact with the ventricular septum. Guidewire 1100 is substantially identical to guidewire 800, with the main difference being that leading end 1120 does not follow a substantially single straight line as it approaches transition section 1130 and double loop 1110. As shown in FIG. 11, the leading end 1120, as it approaches the double loop 1110, bends or curves in a direction away from transition section 1130, and then bands or curves back toward transition section 1130. This configuration of leading end 1120 may help to keep devices delivered over the guidewire, such as a portion of delivery device 10, away from contacting the ventricular septum as well.

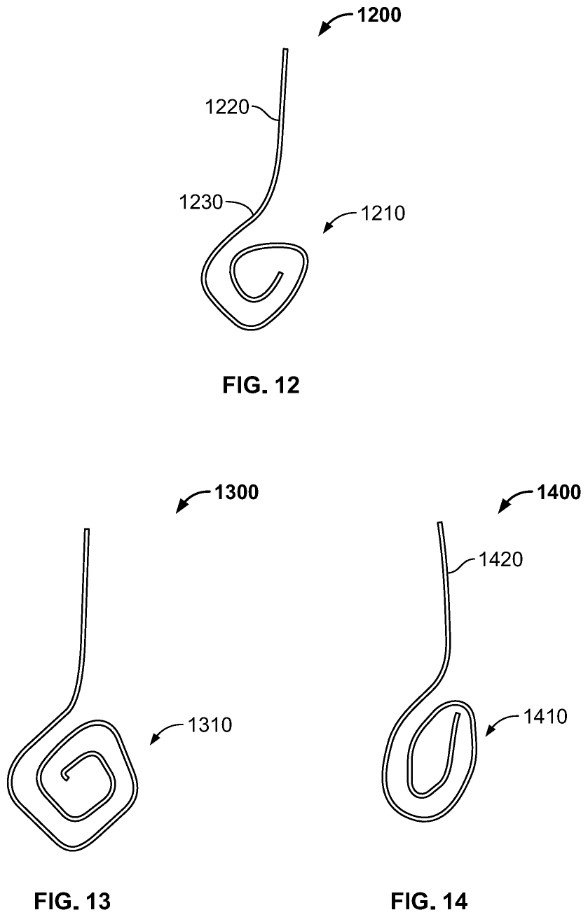

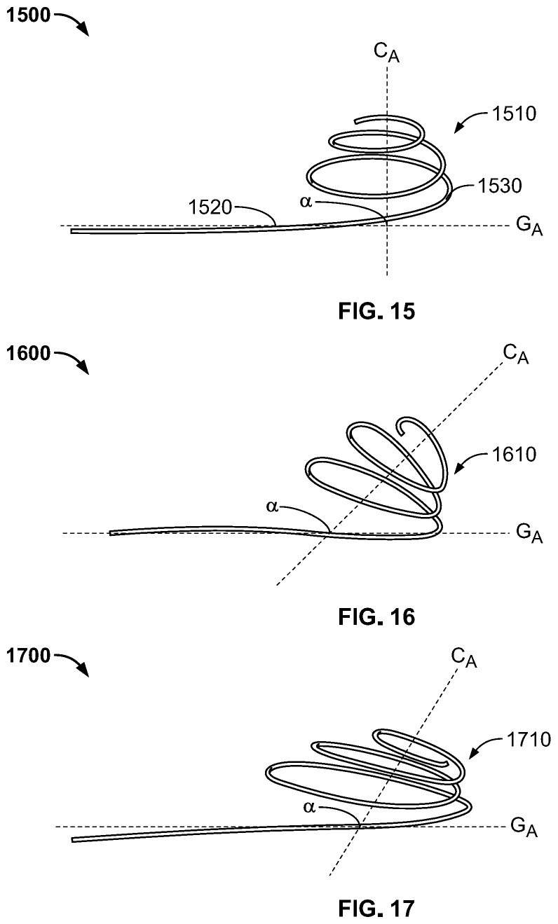

FIG. 12 illustrates a guidewire 1200 that is substantially similar to guidewire 800, with the main differences being the shape of transition section 1230 and double loop 1210. For example, transition section 1230 is less rounded than the transition section 830 of guidewire 800. In other words, the transition section 1230 between double loop 1210 and leading end 1220 is mostly straight with relatively less curvature than the transition section 830 of guidewire 800. In addition, the outer loop portion of double loop 1210 may also include more acute bends instead of a relatively smooth curvature around the outer loop as shown for double loop 810. The bottom or distal end portion of guidewire 1200 (or double loop 1210) may thus have a diamond shape. This configuration of transition section 1230 and double loop 1210 may provide additional assistance in avoiding contact with the ventricular septum, as well as helping the double loop 1210 seat better into the apex of the ventricle. FIG. 13 illustrates a guidewire 1300 that is substantially identical to guidewire 1200, with the main difference being that the inner loop of double loop 1310 also includes relatively sharp bends instead of a relatively smooth curvature around the inner loop. In other words, the entirety of the double loop 1310 has a general diamond-shape, whereas only the bottom of double loop 1210 has a diamond shape. As with guidewire 1200, this configuration of guidewire 1300 may provide additional assistance in avoiding contact with the ventricular septum, as well as helping the double loop 1310 seat better into the apex of the ventricle. The additional structure forming a diamond shape in double loop 1310 may alter the point where the guidewire 1300 transitions from straight to curved relative to the bottom or distalmost portion of the guidewire. The full diamond shape may have result in the wire of the guidewire 1300 at or near the double loop 1310 being longer and having more total wire, compared to a half-diamond shape, which my help better fill the ventricle to stabilize the wire, particularly if the double loop 1310 is provided with a three-dimensional shape similar to those described below in connection with FIGS. 15-17. FIG. 14 illustrates a guidewire 1400 that is substantially similar to guidewire 800, with the main exception being that double loop 1410 is relatively oval or elliptical instead of substantially circular as shown for double loop 810. In other words, the width of double loop 1410 (in a direction transverse the direction of the leading end 1420) is less than the length of the double loop (in a direction parallel to the direction of the leading end). The ratio of the length to the width of double loop 1410 may be greater than 1:1, including for example about 1.5:1, about 2:1 or greater. The configuration of the elongated double loop 1410 may further assistance in avoiding contact with the ventricular septum, as well as helping the double loop 1410 seat better into the apex of the ventricle.

FIGS. 15-17 show additional embodiments of guidewires according to the disclosure. However, it should be understood that the features described in connection with FIGS. 15-17 may be applied to any of the guidewires described above, and the features of any of the guidewires described above may be applied to the guidewires described in connection with FIGS. 15-17. Whereas FIGS. 8-14 illustrate front views of guidewires, FIGS. 15-17 illustrate side views of guidewires. And although the guidewires of FIGS. 8-14 may be flat, so that the entire structure of the guidewire lies within a single plane, the three-dimensional qualities of the guidewires of FIGS. 15-17 may be applied to any of the guidewires of FIGS. 8-14. Similarly, although the guidewires of FIGS. 15-17 are not shown in a front view, the guidewires of FIGS. 15-17 may include any of the shapes shown and/or described in connection to FIGS. 8-14, even if not explicitly shown in FIGS. 15-17.

FIG. 15 illustrates guidewire 1500 that may have the general shape of any of the guidewires described above, including those in FIGS. 8-14. However, as should be clear from the side view of FIG. 15, double loop 1510 has a corkscrew or helical type of shape extending in a direction away from the leading end 1520 of guidewire 1500. More particularly, the leading end 1520 extends along guidewire axis G_{A} (and/or along a loop axis L_{A}) similar to the configuration shown in FIG. 6), and as the guidewire transitions along transition section 1530 to double loop 1510, the loop successively coils away from guidewire axis G_{A}, so that the various loops of double loop 1510 are not positioned within the same plane (or otherwise are positioned at different elevations). In the illustrated embodiment, the terminal tip of the double loop 1510 is positioned farthest away from guidewire axis G_{A} (and/or loop axis L_{A}), although such a feature is not required, and it may be preferable in some embodiments to have the terminal tip of the double loop 1510 point back toward guidewire axis G_{A} (and/or loop axis L_{A}), for example to direct the tip away from contact with the anatomy. The double loop 1510 of guidewire 1500 may coil around a coil axis C_{A}, with the coil axis having an angle α of about 90 degrees, or substantially perpendicular to the guidewire axis G_{A} (and/or loop axis L_{A}). FIGS. 16 and 17 illustrate guidewires 1600, 1700 that are substantially identical to guidewire 1500, with the main difference being the angles α of the coil axes C_{A} relative to the guidewire axes G_{A} (and/or loop axes L_{A}). For example, in FIG. 16, the coil axis C_{A} of double loop 1610 has an oblique angle α relative to the guidewire axis G_{A} (and/or relative to the loop axis L_{A}). For example, the coil axis C_{A} of double loop 1610 relative to the guidewire axis G_{A} (and/or relative to the loop axis L_{A}) may be about 135 degrees. The double loops 1610 of guidewire 1600 may be formed so that each loop has a similar diameter and/or size. Guidewire 1700 may be similar to guidewires 1500 and 1600, with one difference being that the coil axis C_{A} of double loop 1710 relative to the guidewire axis G_{A} (and/or relative to the loop axis L_{A}) is between those shown for guidewire 1500 and guidewire 1600. For example, the angle α between the coil axis C_{A} of double loop 1710 and the guidewire axis G_{A} (and/or the loop axis L_{A}) is between about 90 degrees and about 135 degrees, for example between about 110 degrees and about 115 degrees. Further, whereas the loops of double loop 1610 may each be similar in size and/or diameter, the double loop 1710 of guidewire 1700 may include loops that have decreasing diameters and/or sizes toward the distal tip of the guidewire 1700. It should be understood that other angles between the coil axis C_{A} and the guidewire axis G_{A} (and/or the loop axis L_{A}) may be other angles than those described above. The general configuration of the double loops of the guidewires of FIGS. 15-17, in which the double loop takes on a more three-dimensional shape compared to if the double loop was substantially or entirely within a single plane, may provide certain benefits. For example, these three-dimensional loop shapes may help the loops better fit into the ventricular cavity, and help prevent the wire of the loop from rotating or otherwise moving in non-stable ways. In other words, the three-dimensional loop shapes may provide better overall stability to the wire loop structure, helping ensure that the loop remains in the intended position(s) and orientation(s) during use. Further, as should be understood from the above, the number of coils or loops of the double loop (which, as noted above, need not be limited to two loops), the diameter of the loops (whether the loops have the same or different diameters), the shape of the loops, and the angle (if any) that the loops extend, may all be adjusted as desired to influence the ability of the guidewire to adapt to the shape of the ventricle and thus to increase stability of the guidewire while within the ventricle, preferably while still keeping the guidewire out of contact with the ventricular septum.

As should be understood from the above, typical guidewires of the prior art, when passed through the aortic arch into the left ventricle, are inherently biased toward the outer curve of the aortic arch, increasing the likelihood that those guidewires will contact and/or lie against the membranous septum and/or the ventricular septum. Many of the guidewires described herein are adapted to avoid or otherwise reduce such contact. Many of the guidewires described herein may additionally or alternatively help position the guidewire through a central portion of the native aortic valve annulus (or other valve annulus, e.g. pulmonary valve annulus, depending on the particular trajectory of the guidewire), so that the guidewire is coaxial (or substantially coaxial) with the valve annulus. Such positioning may assist other devices, such as a prosthetic heart valve delivery device, to also be positioned coaxial or substantially coaxial with the native valve annulus. In this scenario, a prosthetic heart valve delivered via a coaxially positioned valve delivery device may lead to a more uniform opening or expansion of the prosthetic valve into the native valve annulus, while also helping reduce conduction system interference and/or arrhythmias from contact with the membranous septum and/or ventricular septum. It may also be desirable to include an atraumatic tip on a distal portion of the guidewire, including the loops described above and/or other atraumatic features, in order to mitigate tissue trauma, for example from contact between the guidewire and the ventricular apex or ventricular septum. Although many of the embodiments described above may achieve one or more of these objectives, additional embodiments are described below.

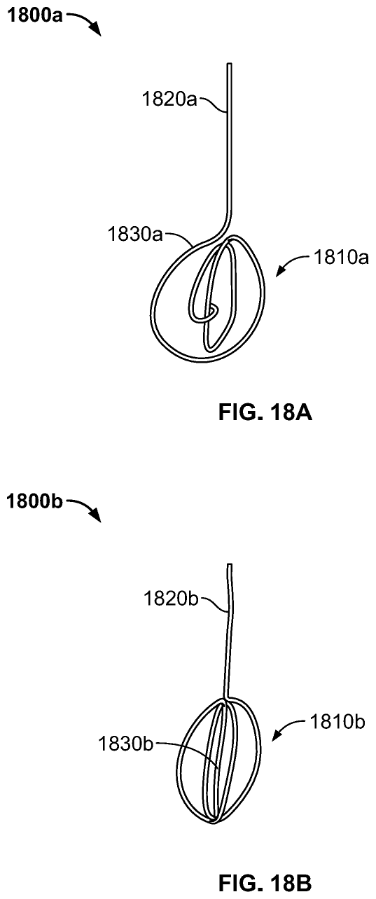

FIG. 18A illustrates another guidewire 1800a that may be substantially similar to other guidewires described herein, with certain differences described in greater detail below. For example, guidewire 1800a may include a double loop 1810a, a leading section 1820a, and a transition section 1830a. The leading section 1820a and transition section 1830a may be substantially similar to those described above in various embodiments, for example with the transition section helping the leading section 1820a extend along an axis that passes through a center portion of double loop 1810a. However, double loop 1810a may be formed with a three-dimensional ball or generally spherical shape. As noted above, the term “double loop” does not require exactly two loops, but rather indicates a generally looping atraumatic structure. For example, double loop 1810a in one embodiment may include a first loop extending generally in a first plane in a generally circular shape, and that first loop may transition into a second loop extending in a second plane in a generally circular shape, the first plane being transverse to the second plane. In the illustrated embodiment, double loop 1810a is formed from a single wire, although that is not required. FIG. 18B illustrates a similar guidewire 1800b that may be substantially similar to guidewire 1800a, with certain exceptions described below. For example, although lead section 1820b is the same as lead section 1820a, the double loop 1820b may be formed of more than one wire so that the double loop may be actuated. For example, guidewire 1800b may include a transition section 1830b that is a continuation of leading section 1820b along the central guidewire axis extending to a terminal distal end of the guidewire, with the transition section 1830b being positioned generally at the center of double loop 1810b. Double loop 1810b may be generally similarly shaped as double loop 1810a, with double loop 1810b having a three-dimensional ball or spherical shape. However, instead of a single wire forming the double loop 1810b, guidewire 1800b may include two, four, or more wires extending from the distal end of the guidewire proximally back toward the point where leading section 1820b and transition section 1830b meet. In one example, double loop 1810b includes two wires extending proximally from the distal tip to form together a generally circular shape in substantially the same first plane, and two additional wires extending proximally from the distal tip to form together a generally circular shape in substantially the same second plane, the first plane being transverse the second plane. These four wires may be fixed to the distal tip of the guidewire 1800b, with the proximal ends being slideable relative to leading section 1820b (and/or transition section 1830b). With this configuration, the leading end 1820b (which may also be referred to as the center wire in this embodiment) may be pulled proximally to actuate the double loop 1810b, the actuation causing the four wires of the double loop 1810b to expand outwardly. In other words, as the leading end 1820b is pulled proximally, the proximal ends of the four wires of double loop 1820b move relatively closer to the distal tip of guidewire 1800b, causing the four wires of the double loop 1810b to bow outwardly to form a more pronounced spherical or ball shape. In one embodiment, the leading section 1820b may be hollow with the transition section 1830b being part of a separate wire extending through the leading section 1820b. In this embodiment, the distal ends of the wires forming the double loop 1810b are fixed to the distal end of the transition section 1830b, while the proximal ends of the wires forming the double loop 1810b are fixed to the distal end of the leading section 1820b. Thus, as the transition section 1830b is pulled proximally through the leading section 1820b, the distance between the distal ends of transition section 1830b and the leading section 1820b decreases, which forces the wires of the double loop 1810b to bow outwardly. When using the guidewire 1800b, the double loop 1810b may be actuated at any point along the delivery, including just prior to entering the aortic arch, or while inside the aortic arch. As with embodiments described above, the double loops 1810a, 1810b, may be shaped using heat setting and/or shape memory properties of the material, although the shapes may be formed using any other suitable modality. In particular, because double loop 1810b can be manually actuated to change shapes, the double loop 1810b may obtain its shape with or without shape memory properties and/or heat setting. Also as with other embodiments described herein, the position of the leading sections 1820a, 1820b with respect to the double loops 1810a, 1810b may (i) help center the guidewires 1800a, 1800b within the native valve annulus during delivery; (ii) help avoid contact between the guidewires 1800a, 1800b with tissue such as the ventricular septum; and/or (iii) reduce the likelihood of damaging native tissue.

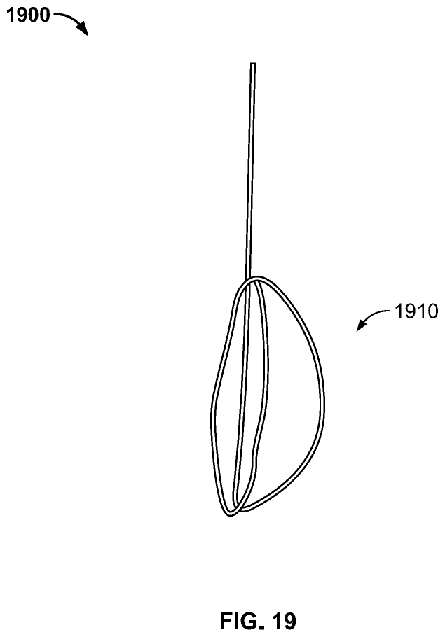

FIG. 19 illustrates a guidewire 1900 that may be substantially similar or identical to guidewires 1800a, 1800b, with certain exceptions described below. Namely, the double loop 1910 of guidewire 1900 may have a shape that forms a portion of a sphere. In particular, double loop 1910 may include two wires that form a generally circular shape in a first plane, and a third wire that forms a generally half-circle shape in a second plane transverse the first plane. This may form a directional bulge, illustrated on the right side of the double loop 1910 in the view of FIG. 19. The double loop 1910 may be capable of actuation, similar to guidewire 1800b, or may be formed of a single wire, similar to guidewire 18000b. In other words, if the double loop 1910 can be actuated, a center wire may extend through a center portion of double loop 1910, which may be pulled proximally to cause the wires of double loop 1910 to bulge. On the other hand, if guidewire 1900 is formed of a single wire, the center wire may be omitted. This asymmetric shape of double loop 1910 may provide the user of guidewire 1900 the ability to orient the double loop 1910 in different orientations to achieve different positioning relative to the native anatomy. It should be understood that a torqueing mechanism may be combined with the asymmetric shape of double loop 1910 in order to customize the amount of centering based on a patient's particular anatomy. In other words, the bulge of the double loop 1910 may be oriented in different directions via torqueing the guidewire to provide an ability to center the guidewire based on how the bulge interacts with the patient's specific anatomy.

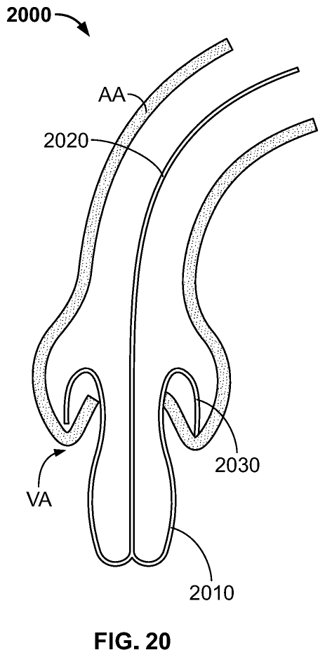

FIG. 20 illustrates a highly schematic side view of another guidewire 2000 positioned within a native valve annulus. In this particular embodiment, guidewire 2000 is illustrated extending through the aortic arch AA and through the native aortic valve annulus VA. Guidewire 2000 may include a leading section 2020 that extends distally toward a distal end of the guidewire 2000, the leading section 2020 transitioning into one or more anchor sections 2010 that extend back proximally, the anchor sections 2010 including anchor tips 2030 that again extend distally. Although guidewire 2000 may include one or more anchor sections 2010, it may be preferable for the guidewire 2000 to include the same number of anchor sections 2010 as the number of native valve leaflets of the valve annulus VA in which the guidewire 2000 will be placed. For example, the aortic valve annulus VA includes three leaflets, and thus the embodiment of guidewire 2000 illustrated in FIG. 20 includes three anchor sections 2010, although only two are visible in the view of FIG. 20. The anchor sections 2010 are preferably substantially equidistantly spaced from the leading section 2020, although that spacing is not required. With this configuration, as the guidewire 2000 is advanced distally through the aortic arch AA and the native valve annulus VA, the anchor tips 2030 will contact the downstream side of the native valve leaflets and/or tissue structure within the sinus of Valsalva. The contact between the anchor tips 2030 and the native tissues helps center the leading section 2020 substantially coaxial with the native valve annulus VA. Preferably, the distance between the distal end of the leading section 2020 and the anchor tips 2030 is large enough to allow a delivery device overlying the guidewire 2000 to extend a desired distance into the left ventricle. In some embodiments, the guidewire 2000 may be integrated with a separate delivery system, such as a delivery device for a prosthetic heart valve. In use, once the guidewire 2000 is in the position illustrated in FIG. 20, a delivery device containing a prosthetic heart valve can be advanced over the leading section 2020 until the delivery device is centered within the native valve annulus VA, at which point the prosthetic valve may be deployed into the native valve annulus VA. In the illustrated embodiment, the risk of contact of the guidewire 2000 with the ventricular septum is reduced at least because the distal end of the guidewire 2000 may be substantially suspended within the interior volume of the left ventricle. Although guidewire 2000 is illustrated being sued in the native aortic valve annulus, it should be understood that a similarly structured guidewire may be used in other heart valves, with possible changes based on the particular valve. For example, if being used in the mitral valve, it may be preferable to include two anchor sections 2010 corresponding to the two native leaflets of the mitral valve.

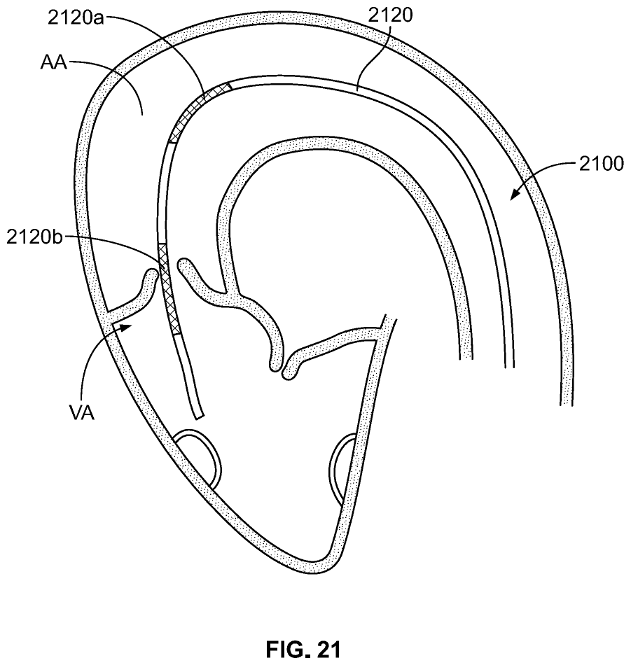

FIG. 21 illustrates a highly schematic side view of another guidewire 2100 positioned within a native valve annulus. In this particular embodiment, guidewire 2100 is illustrated extending through the aortic arch AA and through the native aortic valve annulus VA. Guidewire 2100 may include sections of variable stiffness sections which may assist in centering the guidewire within the native valve annulus VA and/or to help keep the guidewire out of contact with the ventricular septum. In the illustrated embodiment, guidewire 2100 may include a leading section 2120 that is adapted to curve around the aortic arch AA and extend toward, into, or through the native valve annulus VA. The leading section 2120 may include a first low stiffness zone 2120a positioned in a location which is expected to positioned within the aortic arch AA (or another similarly tortuous vessel if another delivery approach is being utilized) when the guidewire 2100 is at or near its intended final position. It should be understood that “low stiffness” may refer to a lower stiffness relative to other portions of the guidewire 2100. This first low stiffness zone 2120a may facilitate the guidewire 2100 in bending or otherwise navigating a tortuous pathway such as the aortic arch AA. The low stiffness of low stiffness zone 2120a may thus also help the portions of guidewire 2100 distal to the low stiffness zone more easily be centered in or through the native valve annulus VA. The leading section 2120 may also include a second high stiffness zone 2120b positioned in a location which is expected to positioned within the native aortic valve annulus VA and/or adjacent the left ventricular outflow tract (“LVOT”) when the guidewire 2100 is at or near its intended final position. It should be understood that “high stiffness” may refer to a higher stiffness relative to other portions of the guidewire 2100. In other words, the guidewire 2100 may have a nominal stiffness along much or most of its length, with the low and high stiffness zones having lower and higher stiffness, respectively, relative to the nominal stiffness. The high stiffness zone 2120b may serve as a rail over which another device, such as a delivery sheath of a prosthetic heart valve delivery device, may slide. The higher stiffness in high stiffness zone 2120b may provide extra stability to the delivery device during delivery toward, into, or across the native valve annulus VA. It should be understood that the variable stiffness may be provided as a constant or inherent feature of the guidewire 2100. For example, the low stiffness zone 2120a and high stiffness zone 2120b may be created via differing material properties of the guidewire 2100, including for example different materials, additional materials (e.g. extra layers for increased stiffness), or other configurations (e.g. slits or cut-outs to reduce stiffness). In other embodiments, the variable stiffness may be provided as a selectable, temporal, and/or actuatable feature. For example, the guidewire 2100 may have a substantially constant stiffness along its length, with stiffness in certain zones being increased or decreased via user input. In one example, an additional stiffening sheath (not illustrated) may be slid over certain portions of the guidewire 2100, such as high stiffness zone 2120b, where it is desired to increase the stiffness of the guidewire. In other examples, electrical current may be passed through guidewire 2100 to vary the stiffness. In this example, the guidewire 2100 may be positioned in the desired location, a delivery device may be slid over the guidewire into or near its desired position, and electrical current could be passed through the guidewire 2100 to guide centering of the guidewire and delivery sheath within the native valve annulus VA. The anatomy may also be used as leverage to help further center the guidewire 2100 and any device positioned over the guidewire in the native valve annulus VA. For example, a distal tip of the guidewire 2100 may be pressed against the papillary muscles to further help center the portion of the guidewire 2100 extending through the native valve annulus VA, although other myocardial structures besides the papillary muscles may be used as leverage points. And although not illustrated, it should be understood that guidewire 2100 may include various features of other embodiments described herein, such as double loops, with or without three-dimensional shapes, to further assist in positioning. It should be understood that, in some embodiments, low stiffness zone 2120a may instead be a high stiffness zone, similar to high stiffness zone 2120b. In those embodiments, the high stiffness zone 2120a may include (but need not include) a pre-set shape that tends to pull the distal section toward the inner curvature of the aortic arch to help achieve centering through the native valve.

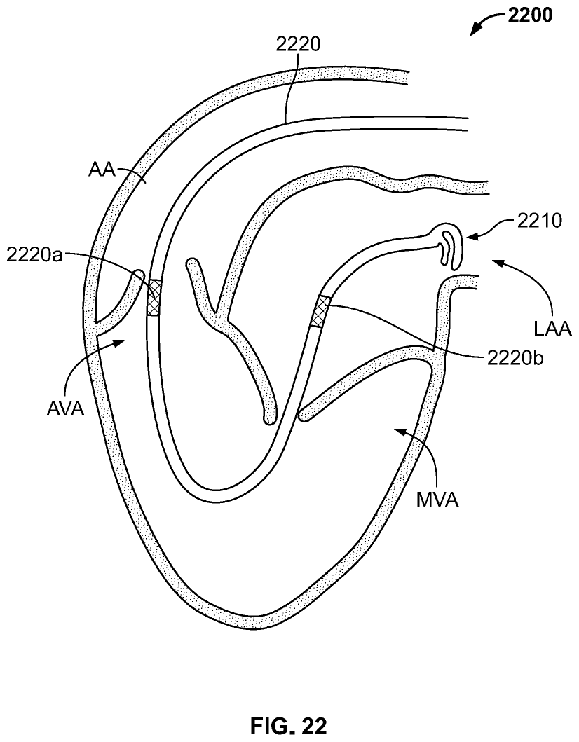

FIG. 22 illustrates a highly schematic side view of another guidewire 2200 positioned within a native valve annulus. In this particular embodiment, guidewire 2200 is illustrated extending through the aortic arch AA, through the native aortic valve annulus AVA, back up through the native mitral valve annulus MVA, and into the left atrial appendage LAA of the left atrium. In the illustrated embodiment, guidewire 2200 includes a first magnetic section 2220a and a second magnetic section 2220b positioned distal to the first magnetic section. The positioning of the magnetic sections may be so that, when the guidewire 2200 is in or near its final intended positioning, the first magnetic section 2220a is positioned within or close to the native aortic valve annulus AVA, and the second magnetic section 2220b is positioned elsewhere within a sufficient distance to interact with the first magnetic section. In the particular illustrated embodiment, the second magnetic section 2220b is positioned with thin left atrium when the first magnetic section 2220a is positioned within the native aortic valve annulus. However, it should be understood that the first magnetic section 2220a may be positioned within any valve annulus where centering is desired, and the second magnetic section 2220b may be positioned anywhere else that can affect the first magnetic section through magnetic attraction (or repulsion). In some embodiment, one or both magnetic sections 2220a, 2220b are permanent magnets. However, it may be preferable for one or both magnetic sections 2220a, 2220b to be capable of activation, for example via electrical current applied to the magnetic sections, so that the magnetic sections only interact when the user inputs electric current to activate the magnets. Preferably, the positioning of the second magnetic section 2220b in relation to the first magnetic section 2220a is such that the second magnetic section is positioned in an area opposite where the first magnetic section would tend to be biased toward. In the illustrated example, the portion of the leading end 2220 of guidewire 2200 near the aortic valve annulus AVA would tend to be biased to the left of the illustration, and thus the second magnetic section 2220b is positioned to the right of the first magnetic section 2220a, so that, upon activation of the magnets, the first magnetic section would be pulled against the biasing force toward the center of the aortic valve annulus AVA. Although not required, the guidewire 2200 may also include a distal fixation member 2210. In the illustrated embodiment, the distal fixation member 2210 may take the form of a braided mesh, which may have shapes or configurations similar to Amplatzer occluder devices offered by Abbott Vascular, or other left atrial appendage LAA closure devices. With this configuration, the distal fixation member 2210 may be positioned within the left atrial appendage LAA to temporarily stabilize the guidewire 2200, for example when the magnetic sections 2220a, 2220b are activated, so that the first magnetic section tends to be pulled toward the second magnetic section. It should be understood that the distal fixation member 2210, if included, may take any suitable form, and may be suited to the particular delivery location. For example, instead of positioning the distal end of the guidewire 2200 in the left atrial appendage LAA, it may be instead be positioned in a pulmonary vein (not illustrated) for temporary securement. If positioned in the pulmonary vein, it may be preferable for the distal fixation member 2210 to take the shape of a stent, for example a generally cylindrical stent that can temporarily stabilize the distal end of the guidewire 2200. It should be understood that guidewire 2200 may be similarly used in the right side of the heart instead of the left side of the heart as illustrated. The ability to temporarily fix or stabilize the distal end of the guidewire 2200 while pulling (or pushing) the first magnetic section 2220a toward the center of the native aortic valve annulus AVA, the guidewire can be centered within the valve annulus and also avoid contact with the ventricular septum. As with other embodiments, it should be understood that features of other guidewires described herein may be combined with those of guidewire 2200 where suitable.

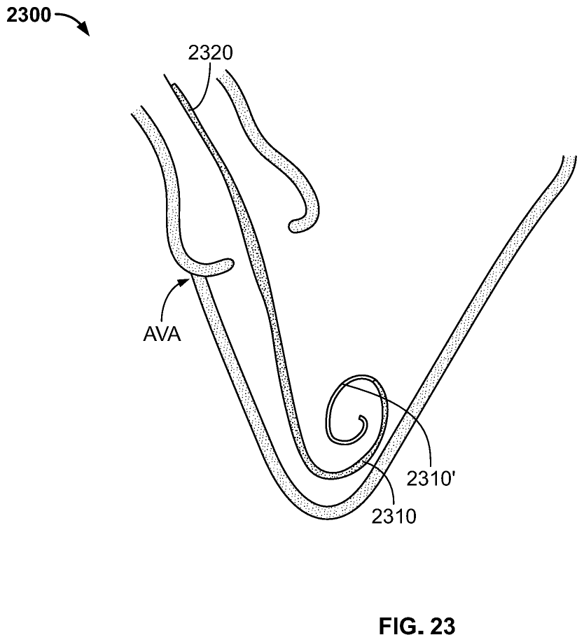

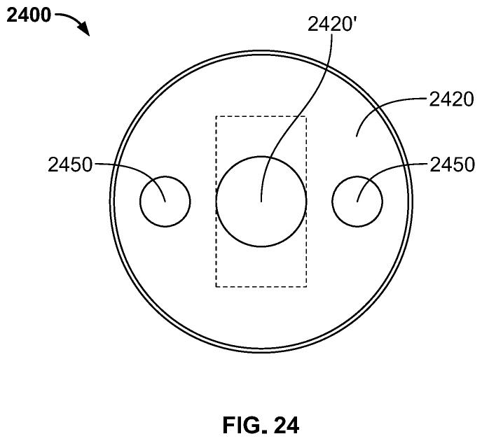

FIG. 23 illustrates a highly schematic side view of another guidewire 2300 positioned through a native valve annulus and into a ventricle. In this particular embodiment, guidewire 2300 is illustrated extending through the native aortic valve annulus AVA and into the left ventricle. In the illustrated embodiment, guidewire 2300 includes a leading end 2320 that may include a partial loop 2310 at a terminal distal end thereof. In the illustrated embodiment, partial loop 2310 forms a “J” shape, a “U” shape, or a generally semi-circular shape. Guidewire 2320 may be a two-part guidewire. For example, the leading end 2320 may be a first guidewire, a microcatheter, or the like with a hollow interior leading to an open terminal distal end. The guidewire 2320 may include a second guidewire, such as an inner core, which may be for example a metal wire or more traditional guidewire, adapted to pass through the first guidewire or microcatheter. The second guidewire or inner core may include a loop 2310′ at its distal end, which may be similar to any of the double loops described above. In one embodiment, the outer guidewire, including leading end 2320, may have as stiffness that is greater than the stiffness of the inner cord or second guidewire. With this configuration, the two guidewire system may form a telescoping guidewire, with the greater stiffness of the outer guidewire helping to stabilize the guidewire system in the left ventricle, which may help center the guidewire within the native aortic valve annulus AVA and/or help reduce motion of the guidewire, which may in turn reduce potential for tissue trauma. It should be understood that, by including both the inner core and the outer guidewire, instead of merely providing a single guidewire with the illustrated shape and configuration, it may be possible to better customize the level of centering and/or to mitigate potential for conduction system issues with the telescoping action.

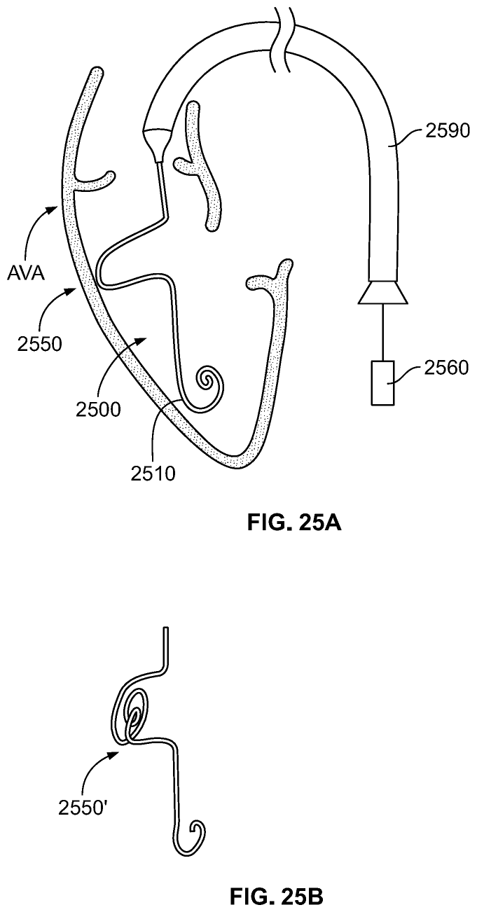

FIG. 25A is a highly schematic cross section of a prosthetic heart valve delivery device 2590 having a distal end positioned adjacent the native aortic valve annulus AVA. Prosthetic heart valve delivery device 2590 may be similar or identical to delivery device 10, with certain differences described in greater detail below. A guidewire 2500 is also shown passing through the native aortic valve annulus AVA and into the left ventricle, with the delivery device 2590 having been passed over portions of the guidewire 2500. Guidewire 2500 may include a loop 2510 at its distal end, and the loop may be a traditional guidewire loop or any of the loops described herein. Guidewire 2500 may include a “C”-shaped, “U”-shaped, or other similar bend 2550 proximal to the loop 2510. Preferably, the bend 2550 is located at a position along the guidewire 2500 so that, when the guidewire is at or near its final desired position, for example with the loop 2510 near or adjacent the ventricular apex, the bend 2550 is positioned within the ventricle (e.g. the left ventricle) distal the native valve annulus (e.g. the native aortic valve annulus AVA). In the illustrated embodiment, the bend 2550 is oriented so that the apex of the bend contacts tissue of the ventricular septum, resulting in the portion of the guidewire just proximal to the bend being centered within the native valve annulus AVA. Thus, the tip of delivery device 2590 will also be positioned centered within the native valve annulus 2550. In some embodiments, the guidewire 2500 may include a torque control member 2560, for example a handle or similar device at a proximal end of the guidewire 2500. The torque control member 2560 may be rotated in order to rotate the bend 2550 about an axis. The axis of rotation may be defined by the main portion of the guidewire 2500 proximal to the bend 2550. When the guidewire 2500 is at or near its final position, it is possible that the bend 2550 will not be oriented as desired. Thus, the torque control member 2560 may be actuated (e.g. rotated) to re-orient the bend 2550 to contact the native anatomy in a desired position and/or orientation to ensure that the guidewire 2500 is centered within the native valve annulus AVA. However, the torque control member 2560 may be omitted in some embodiments. The bend 2550 is illustrated in FIG. 25A as having a substantial “two-dimensional” shape. In other words, the entirety of bend 2550 may be positioned substantially within a single plane. However, as illustrated in FIG. 25B, an alternate embodiment of the bend 2550′ may include a “three-dimensional” shape. In other words, bend 2550′ is bent so that the bend is not positioned in a single plane. The three-dimensional bend 2550′ may provide greater control and/or ability to center the guidewire 2500, particularly if torque control member 2560 is included. The three-dimensional bend 2550′ may also be suited so that, where there is contact with anatomical structures vulnerable to conduction interference, that contact is provided with low pressure to minimize the likelihood of any such conduction interferences. It should be understood that, with bend 2550 (or bend 2550′, or a similar end), the portion of the guidewire 2500 distal to the bend may be completely traditional, similar to that shown in FIG. 4, while still allowing for centering of the guidewire.

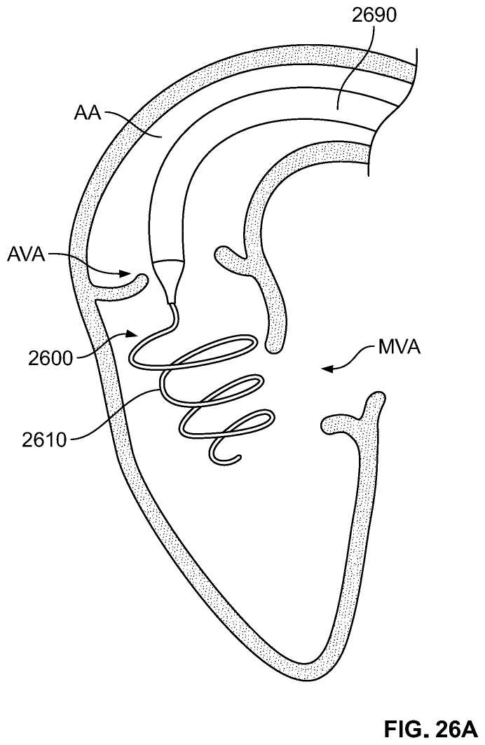

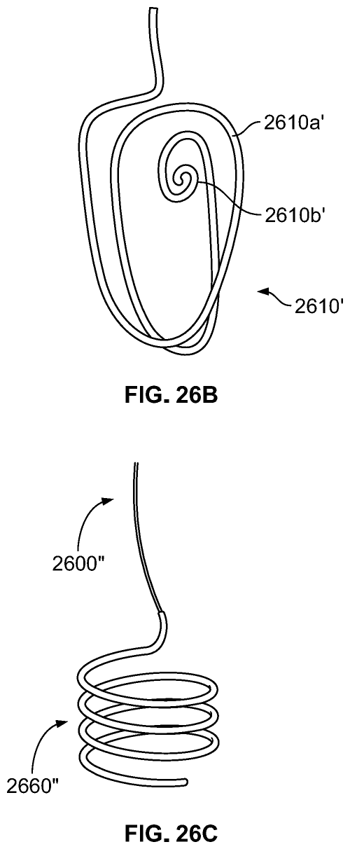

FIG. 26A is a highly schematic cross section of a prosthetic heart valve delivery device 2690 having a distal end positioned adjacent the native aortic valve annulus AVA. Prosthetic heart valve delivery device 2690 may be similar or identical to delivery device 10 and/or 2590. A guidewire 2600 is also shown passing through the native aortic valve annulus AVA and into the left ventricle, with the delivery device 2690 having been passed over portions of the guidewire 2600. Guidewire 2600 may include a loop 2610 at its distal end. Loop 2610 may include one or more loops. In the illustrated embodiment, loop 2610 includes three to four loops that extend in a corkscrew-type fashion, with the diameter of each loop decreasing toward the distal end of the guidewire 2600. However, in other embodiments, the diameter of the loops may be about the same, or otherwise may even increase toward the distal end of the guidewire. The loops 2610 may generally circle around an axis that is substantially aligned with the portion of the guidewire 2600 proximal to the loops (not illustrated in FIG. 26A). With this configuration, the loop 2610 may be particularly suited to rest within the apex of the ventricle (left ventricle or right ventricle) to stabilize the portion of the guidewire 2600 that extends through the native valve annulus (e.g. the aortic valve annulus AVA) at a center of the valve annulus. The shape and configuration of loop 2610 may also help mitigate any trauma to the native tissue during insertion of the guidewire 2600 into the heart. FIG. 26B illustrates an alternate embodiment of the loop 2610′ of guidewire 2600, in a view looking down through the loop toward the distal end of the loop. In particular, loop 2610′ may include a first portion 2610a′ with relatively high stiffness, and a second portion 2610b′ with relatively low stiffness. The relatively high stiffness portion 2610a′ may be positioned closer to the proximal end of the loop 2610′, which may help provide greater force to stabilize the guidewire 2600 (and delivery device 2690) within a center of the native valve annulus AVA. The relatively low stiffness portion 2610b′ may be positioned closer to the distal end of the loop 2610′, which may help reduce trauma to the native tissue that the distal loop contacts. The variable stiffness may be created by any suitable means, including thicker or thinner sections of guidewire material. Although FIGS. 26A-B illustrate the helical portion of the guidewire 2600 being formed by the guidewire itself, in other embodiments, a separate member may be provided to force the guidewire to take the helical, coiled, or looped shape. For example, FIG. 26C illustrates guidewire 2600″, which may be a substantially straight guidewire. A separate telescoping shaft 2660″ may be provided which may be advanced over the guidewire 2600″ near the distal end of the guidewire. This telescoping effect may allow the separation of the advancement of the guidewire 2600″ and the shaping of the guidewire to the desired helical, coiled, or looped shape. The telescoping approach may also allow the stiffness of the combined assembly to be varied in any desirable fashion. The telescoping shaft 2660″ may have a helical shape similar to that of loop 2610. In other words, telescoping shaft 2660″ may include loops or coils, and those loops or coils may decrease in diameter toward the distal end. Similar to loop 2610′, the telescoping shaft 2660″ may include a relatively stiff proximal end to better help center the guidewire 2600″ within the native valve annulus, and a relatively soft distal end to help minimize tissue trauma.

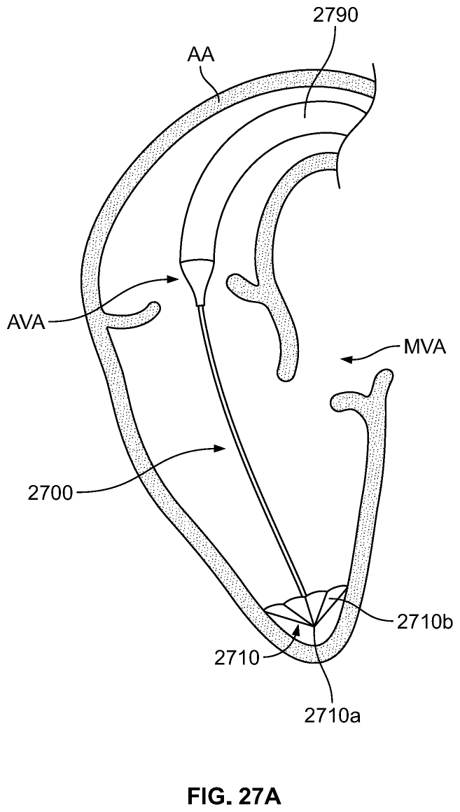

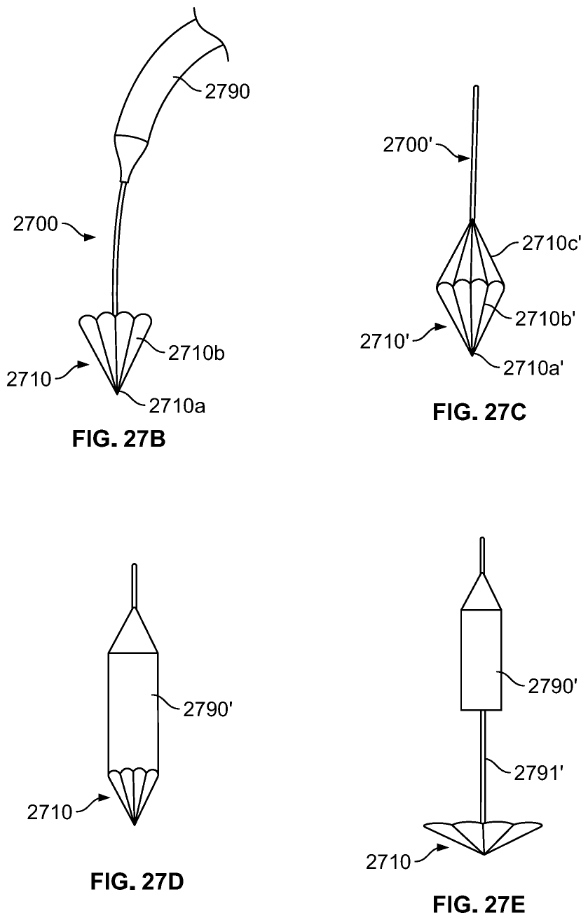

FIG. 27A is a highly schematic cross section of a prosthetic heart valve delivery device 2790 having a distal end positioned adjacent the native aortic valve annulus AVA. Prosthetic heart valve delivery device 2790 may be similar or identical to delivery device 10 and/or 2590. A guidewire 2700 is also shown passing through the native aortic valve annulus AVA and into the left ventricle, with the delivery device 2790 having been passed over portions of the guidewire 2700. In this embodiment, guidewire 2700 may be a single relatively straight wire along an entire length thereof, with the guidewire terminating in an anchor or distal tip 2710. In other embodiments described herein, the distal tip of the guidewire is typically a coiled loop formed from part of the guidewire. However, in this embodiment, the distal tip 2710 may be a separate structure coupled to the distal end of the guidewire 2700. In the embodiment of FIG. 27A, distal tip 2710 may be formed of a soft material, such as a polymer or elastomer to provide an atraumatic point of contact with native tissue. Further, distal tip 2710 may include a central portion 2710a and a plurality of extensions 2710b extending therefrom, each extension being spaced apart from one another circumferentially. In this particular illustrated embodiment, distal tip 2710 forms a shape similar to a shuttlecock, with the distalmost central portion 2710a being generally convex and intended to contact tissue within the ventricular apex, and the extensions 2710b extending proximally and radially outward from the central portion. During delivery of the guidewire 2700, the extensions 2710b may extend mostly or fully proximally from the central portion 2710a, without flaring, or only minimally flaring, radially outward. With this configuration, the distal tip 2710 may be collapsed during delivery to maintain a relatively small profile, and once the guidewire 2700 enters the ventricle, the distal tip 2710 may be expanded by allowing the extensions 2710b to flare radially outwardly. For example, FIG. 27B illustrates the distal tip 2710 in a collapsed condition. In the embodiment shown in FIGS. 27A-B, the distal tip 2710 may be passively collapsed and expanded. However, in other embodiments, the distal dip 2710 may be actively collapsed and expanded. For example, as shown in FIG. 27C, distal tip 2710′ includes a central portion 2710a′ and extensions 2710b′ substantially identical to those shown in FIGS. 27A-B. However, distal tip 2710′ may also include a plurality of tines or connections 2710c′ that connect the guidewire 2700′ to extensions 2710b′. An actuation member may extend through the center of guidewire 2700′ and connect to central portion 2710a′, so that advancing or retracting the actuation member relative to the guidewire 2700′ may force the distal tip 2710′ to collapse or expand as desired. In some embodiment, instead of providing distal tip 2710 as part of guidewire 2700, the distal tip may be provided as part of the delivery system. For example, FIG. 27D illustrates a distal sheath of a delivery system 2790′. Typically, a delivery system for a collapsible and expandable prosthetic heart valve includes an atraumatic distal tip at the distalmost end. In the illustrated embodiment, the atraumatic distal tip of delivery device 2790′ is replaced with the distal tip 2710 illustrated in FIGS. 27A-B. During delivery of the delivery device 2790′, the distal tip 2710 is maintained in the collapsed condition, shown in FIG. 27D. Once the delivery device 2790′ is at or adjacent the target site for delivery of the prosthetic heart valve, the distal tip 2710 may be advanced distally, for example by advancing a connecting wire 2791′ that couples the distal tip 2710 to the delivery device 2790′. With the distal tip 2710 advanced, it may passively expand as described above. When the distal tip 2710 expands and is positioned within the apex of the ventricle, it may provide stabilization for centering the delivery device 2790′. The connecting wire 2791′ may be stiff to assist in the stabilization.

According to one embodiment of the disclosure, a guidewire is configured for insertion into a heart, the guidewire comprising:
a proximal end; and
a distal end portion, the distal end portion including (i) a leading section; (ii) a loop structure at a terminal distal end of the guidewire; and (iii) a transition section extending between the leading section and the loop structure,
wherein, in the absence of applied forces, the leading section is not tangential to the loop structure; and/or
in the absence of applied forces, (i) the leading section extends along a guidewire axis; (ii) the loop structure defines a first point at which a first line tangential of the first point is substantially parallel to the guidewire axis; and (iii)the loop structure defines a second point diametrically opposed from the first point, a second line tangential of the second point being substantially parallel to the guidewire axis; and/or
in the absence of applied forces, the guidewire axis is positioned between the first line and the second line; and/or
in the absence of applied forces, the first point and the second point are substantially equidistant from the guidewire axis; and/or
the loop structure includes at least two loops; and/or
the loop structure extends from the transition section to a distal tip of the guidewire; and/or
the loop structure has a helical or corkscrew configuration so that portions of the loop structure lie within a plane that does not pass through the leading section; and/or
the loop structure coils about a coil axis, the coil axis being substantially perpendicular to a guidewire axis along which the leading section extends; and/or
the loop structure coils about a coil axis, the coil axis being oblique to a guidewire axis along which the leading section extends; and/or
the leading section includes a bend; and/or
the loop structure has a length and a width, the length being substantially equal to the width; and/or
the loop structure has a length and a width, the length being between about 1.5 and about 2 times greater than the width; and/or
the loop structure is substantially continuously smoothly curved; and/or
the loop structure includes a plurality of angled bends.

According to another embodiment of the disclosure, a method of positioning a guidewire within a heart comprises:
advancing a distal end portion of the guidewire into a left or right ventricle of the heart until a loop structure at a terminal distal end of the guidewire is seated within the left or right ventricle, the distal end portion of the guidewire including a leading section and a transition section extending between the leading section and the loop structure,
wherein when the loop structure is seated within the left or right ventricle, an entire length of the leading section positioned between a native valve annulus of the heart and the transition section of the guidewire is out of contact with a ventricular septum separating the left ventricle from the right ventricle of the heart; and/or
advancing the distal end portion of the guidewire includes advancing the distal end portion of the guidewire into the left ventricle, by advancing the distal end portion of the guidewire into a femoral artery, around an aortic arch, and through the aortic valve annulus; and/or
advancing a delivery device over the guidewire while the loop structure is seated within the left ventricle, until a distal end portion of the delivery device is adjacent the aortic valve annulus; and/or
retracting a sheath of the delivery device to allow a collapsible prosthetic heart valve positioned within the sheath to expand into contact with the aortic valve annulus.

Although the invention herein has been described with reference to particular embodiments, it is to be understood that these embodiments are merely illustrative of the principles and applications of the present invention. For example, features described in connection with one embodiment may be combined with features described in connection with another embodiment without varying from the scope of the invention. As examples, any of the three-dimensional loop configurations of FIGS. 16-18 may be used with any of the other guidewires described herein, including those of the prior art. Similarly, any of the loop shapes, for example those described in connection with FIGS. 8-15, may be used with any of the three-dimensional configurations of FIGS. 16-18. Still further, other features, such as the bent leading end of guidewire 1100, or the elongated length-to-width ratio of guidewire 1500, may be combined with any of the other guidewire features described herein. It is therefore to be understood that numerous modifications may be made to the illustrative embodiments and that other arrangements may be devised without departing from the spirit and scope of the present invention as defined by the appended claims.

1. A guidewire for insertion into a heart, the guidewire comprising:
a proximal end; and
a distal end portion, the distal end portion including (i) a leading section; (ii) a loop structure at a terminal distal end of the guidewire; and (iii) a transition section extending between the leading section and the loop structure,
wherein, in the absence of applied forces, the leading section is not tangential to the loop structure.

2. The guidewire of claim 1, wherein, in the absence of applied forces, (i) the leading section extends along a guidewire axis; (ii) the loop structure defines a first point at which a first line tangential of the first point is substantially parallel to the guidewire axis; and (iii) the loop structure defines a second point diametrically opposed from the first point, a second line tangential of the second point being substantially parallel to the guidewire axis.

3. The guidewire of claim 2, wherein, in the absence of applied forces, the guidewire axis is positioned between the first line and the second line.

4. The guidewire of claim 3, wherein, in the absence of applied forces, the first point and the second point are substantially equidistant from the guidewire axis.

5. The guidewire of claim 1, wherein the loop structure extends from the transition section to a distal tip of the guidewire, the loop structure having a helical or corkscrew configuration so that portions of the loop structure lie within a plane that does not pass through the leading section.

6. The guidewire of claim 5, wherein the loop structure coils about a coil axis, the coil axis being substantially perpendicular to a guidewire axis along which the leading section extends.

7. The guidewire of claim 1, wherein the loop structure is a single wire and forms a spherical shape in the absence of applied forces.

8. The guidewire of claim 1, wherein the loop structure includes a plurality of wires each having a distal end fixedly coupled to the distal end of the guidewire, and each having a proximal end that is slideable relative to the guidewire, so that upon the proximal ends sliding relative to the guidewire, the plurality of wires being forced to bow outwardly upon sliding of the proximal ends relative to the guidewire.

9. (canceled)

10. A method of positioning a guidewire within a heart, the method comprising:
advancing a distal end portion of the guidewire into a left or right ventricle of the heart until a loop structure at a terminal distal end of the guidewire is seated within the left or right ventricle, the distal end portion of the guidewire including a leading section and a transition section extending between the leading section and the loop structure,
wherein when the loop structure is seated within the left or right ventricle, an entire length of the leading section positioned between a native valve annulus of the heart and the transition section of the guidewire is out of contact with a ventricular septum separating the left ventricle from the right ventricle of the heart.

11. The method of claim 10, wherein advancing the distal end portion of the guidewire includes advancing the distal end portion of the guidewire into the left ventricle, by advancing the distal end portion of the guidewire into a femoral artery, around an aortic arch, and through the aortic valve annulus.

12. The method of claim 11, further comprising advancing a delivery device over the guidewire while the loop structure is seated within the left ventricle, until a distal end portion of the delivery device is adjacent the aortic valve annulus.

13. The method of claim 12, further comprising retracting a sheath of the delivery device to allow a collapsible prosthetic heart valve positioned within the sheath to expand into contact with the aortic valve annulus.

14. A guidewire for insertion into a heart, the guidewire comprising:
a proximal end; and
a distal end portion having a distal fixation member configured to temporarily fix the distal end portion of the guidewire relative to the heart;
a first magnetic section being positioned on the guidewire at a first location along the guidewire; and
a second magnetic section being positioned on the guidewire at a second location along the guidewire a spaced distance from the first magnetic section,
wherein the second magnetic section is configured to pull or push the first magnetic section toward or away from the second magnetic section.

15. The guidewire of claim 14, wherein the distal fixation member is either (i) a braided mesh configured for placement in a left atrial appendage of the heart; or (ii) a stent configured for placement in a pulmonary vein of the heart.

16. The guidewire of claim 14, wherein the first magnetic section and the second magnetic section are electromagnets.

17. A guidewire for insertion into a heart, the guidewire comprising:
a proximal end; and
a distal end portion having a distal tip, the distal tip being formed of a polymer or elastomer, the distal tip having a central portion adapted to contact a ventricular apex of the hear, the distal tip having a collapsed delivery condition and an expanded functional condition.

18. The guidewire of claim 17, wherein the distal tip includes a central portion and a plurality of extensions extending proximally from the central portion, the plurality of central portions being spaced apart from one another in a circumferential direction.

19. The guidewire of claim 17, wherein the distal tip is configured to passively transition between the collapsed delivery condition and the expanded functional condition.

20. The guidewire of claim 17, wherein the distal tip is configured to actively transition between the collapsed delivery condition and the expanded functional condition.
